(19) United States
(12) Patent Application Publication
(10) Pub. No.: US 2013/0261445 A1
(43) Pub. Date: Oct. 3, 2013

(54) REPRESENTATION OF BLOOD VESSELS AND TISSUE IN THE HEART

(71) Applicants: Dirk Ertel, Neunkirchen am Brand (DE); Yiannis Kyriakou, Spardorf (DE)

(72) Inventors: Dirk Ertel, Neunkirchen am Brand (DE); Yiannis Kyriakou, Spardorf (DE)

(21) Appl. No.: 13/855,078

(22) Filed: Apr. 2, 2013

(30) Foreign Application Priority Data
Apr. 2, 2012 (DE) 102012205351.4

(51) Int. Cl. A61B 5/00; A61B 6/03; A61B 8/08; A61B 6/00; A61M 5/00; A61B 5/055

(52) U.S. Cl. CPC A61B 5/0044; A61M 5/007; A61B 5/055; A61B 8/481; A61B 6/481; A61B 6/4441; A61B 6/037; A61B 5/72
USPC 600/431

(57) ABSTRACT

A method for representation of blood vessels and/or tissue in a heart of a human or animal body is described, with an imaging method and an assignment method, which allows the assignment to a heart phase of time-resolved images acquired with the imaging method. A mask series with a series of temporally-resolved first images of the heart is acquired, wherein the first images are assigned to a heart phase by the assignment method. A filling series with a series of temporally-resolved second images of the heart is acquired, wherein the second images are assigned to a heart phase by the assignment method. The first and second images assigned to the same heart phase are computed for creating a series of phase-selective subtraction images. The phase-selective subtraction images and/or the second images are computed, taking into account the heart movement for creating temporally and/or spatially-resolved perfusion data of the heart.

### CROSS REFERENCE TO RELATED APPLICATIONS

This application claims priority of German Patent Application No. 10 2012 205 351.4 DE filed Apr. 2, 2012. All of the applications are incorporated by reference herein in their entirety.

### FIELD OF INVENTION

A method for representation of blood vessels and/or tissue in a heart of a human or animal body, especially a parametric heart throughflow visualization with suitable movement compensation, is provided. Further, a corresponding apparatus for carrying out the method is provided. The method is preferably used in angiographic x-ray devices, for example C-arm angiographs.

### BACKGROUND OF INVENTION

Angiography is a method that is sufficiently well known in medicine for representation of vessels, mostly blood vessels, by means of diagnostic imaging methods such as x-ray or Magnetic Resonance Tomography (MRT). For an improved representation of the vessels under examination, especially for representing them in isolation, above all without disruptive background areas etc., Digital Subtraction Angiography (DSA) has been developed. As in standard angiography a contrast medium, which for example exhibits a high coefficient of the absorption for x-ray radiation and shows the vessels in high contrast on an x-ray image taken simultaneously, is injected into the patient. By contrast with standard angiography methods, in DSA what is referred to as a mask image without contrast medium is recorded before the injection. This is stored and subtracted from what are referred to as the filling images taken after the contrast medium has been added. By this process background structures are eliminated and in the ideal case just the vessels are visible on the differential images.

Related to this method are what are referred to as bolus tracking or perfusion imaging methods, in which the contrast medium is injected as a bolus and subsequently a series of high-resolution images of the area through which the contrast medium is flowing is recorded. From the dynamics of the inflow and outflow of the contrast medium in a vessel or a perfused tissue conclusions can be drawn about the perfusion. The timing curve is generally represented as a time-density curve or time-intensity curve, which represents the intensity value of a specific pixel in an image in the time curve. A Gamma variate function can for example be fitted onto this curve, which facilitates kinetic analysis. From the course of the curve perfusion data such as the blood flow, the blood volume and the mean transit time of the blood through the vessel can be determined. For the brain these methods are at least well proven in theory and described for magnetic resonance tomography for example by Leif Ostergaard et al. in “High Resolution Measurement of Cerebral Blood Flow using Intervascular Tracer Bolus Passages: I. Mathematical approach and statistical analysis.” Magnetic Resonance in Medicine (1996) Vol. 36, No. 5, pages 715 to 725.

The disadvantage is that methods such as DSA and Bolus Tracking are not able to be applied to moving objects such as the heart for example, since the heart has continued to move between the acquisition of the mask image and the acquisition of the filling image. Additional important data of the heart which would be able to be determined quickly and at low cost by means of a DSA, such as throughflow parameters for example, can only be obtained with complex methods such as the use of intravascular ultrasound (IVUS) for example.

A method for creating fused images of a moving organ of the body is known from US 2010/0074504 A1, in which by means of an x-ray diagnostic device, a plurality of mask images with a frame rate of 60 frames per second and subsequently, after introduction of a contrast medium, a contrast or filling image is created. Then the at least one mask image is defined which was created in the same movement phase as the contrast or filling image and is a best match for the latter. These two images are overlaid to form a fusion image. The images are created from pure subtraction image datasets of an arterial and perfusion phase of the heart.

WO 2005/023086 A2 discloses a method for analysis of attributes of blood vessels by developing an atlas with statistical measurements, which has been formed by measurement of at least one feature on different individuals. The measurement of this feature on an individual will be compared with the statistical measurements of the vessel characteristics. Included in the analysis of the vessel characteristics for example are their number, their radius, their number of branches etc. An output signal which displays the physical characteristics then reproduces the result of the comparison.

### SUMMARY OF INVENTION

An object is to provide a parametric heart throughflow visualization with suitable movement compensation, which makes possible the creation of temporally and/or spatially-resolved perfusion data of the heart and provides a representation of said data in a map of the heart.

The object is achieved by a method and by an apparatus as claimed in the independent claims. Further advantages and features emerge from the dependent claims as well as from the description and the enclosed figures.

A method is specified for representation of blood vessels and/or tissue in a heart of a human or animal body with an imaging method and an assignment method which allows time-resolved images acquired with the imaging method to be assigned to a heart phase, wherein the blood in the blood vessels and/or the tissue exhibits two states, wherein the blood in the first state does not have a contrast medium added to it, and wherein the blood in the second state has a contrast medium added to it, comprising the steps:
(a) Acquisition by the imaging method of a mask series comprising a series of temporally-resolved first images of the heart, wherein the blood is in the first state, and wherein the first images d are assigned in each case to a heart phase by the assignment method;
(b) Acquisition by the imaging method of a filling series comprising a series of temporally-resolved second images of the heart, wherein the blood is in the second state and wherein the second images are assigned in each case to a heart phase by the assignment method;
(c) Computation of the respective first and second images assigned to the same heart phase for creating a series of phase-selective subtraction images;
(d) Computation of the phase-selective subtraction images and/or of the second images of the at least one heart cycle, taking into account the heart movement for creating temporally and/or spatially-resolved perfusion data of the heart.

The imaging method can for example be a magnetic resonance tomography method MRT, ultrasound, an x-ray method or a positron emission tomography method. The method must merely be able to deliver images which possess a suitable time resolution in order to be able to represent the throughflow of the contrast medium (referred to as the first pass) sufficiently well resolved, i.e. with a resolution of <300 ms, preferably of <100 ms. Therefore x-ray projection images are especially preferred, since these can be acquired with an especially high-frequency of e.g. 30-100 images per second, preferably 40-80 images per second. Both the images of the mask series and also those of the filling series are recorded with the same image slice, or for x-ray images, from the same projection direction, so that they can be compared to one another as well as possible. Preferably the x-ray images are recorded with a C-arm device.

Preferably the method is carried out during a surgical or angiographic intervention, wherein at least one medical instrument, especially a catheter via which contrast medium is injected and/or if necessary an instrument for carrying out minimally-invasive vascular surgery, is located in the blood vessels. This is however merely the case in some embodiments. The method can also be carried out independently of a surgical intervention.

An electrocardiogram (EKG) is also preferably used for the assignment method. This allows the heart phase to be recorded in each case during the acquisition of an image, and thus images recorded at different points in time can be each assigned to a specific heart phase. Other assignment methods are less preferred, but also conceivable however, for example the determination of the heart phase from the contents of the respective images.

The object is to enable the images created with the imaging method to be assigned temporally to one another by the assignment method on the one hand and on the other hand enable them to be assigned one or more states of the heart. The fact that the first images of the mask series and the second images of the filling series are able to each be assigned to one point in time and by the assignment method also to a corresponding state of the heart—and ultimately also to a certain form of the heart—enables phase-selective subtraction images to be created. These can be included for the creation of the perfusion data of the heart.

Preferably the breath should be held both for the mask series and also for the filling series, also the EKG data is preferably recorded as well for both the mask series and also for the filling series. The injection protocol for the contrast medium is preferably able to be matched to the heart cycle, i.e. to the EKG signal, such that all blood vessels of interest or the tissue of interest are able to be represented on the images. The contrast medium is preferably given as a bolus, wherein the acquisition of the filling series is set temporally such that at least the first pass of the contrast medium through vessels or tissue will be represented. Since it precisely with changes in diseased vessels that it is not precisely known whether the contrast medium may arrive late in the heart, the filling series preferably takes longer than the mask series, for example 5-40 seconds, typically around 10-20 seconds. The contrast medium can either be injected into a vein or preferably into an artery a short distance before the area to be visualized.

Advantageously both the right and also the left coronary artery are able to be selected for the injection of the contrast medium. Also preferred is an injection into the lung artery, through which a visualization and parameterization of an atrium and/or a heart chamber is able to be represented.

Expediently the computation of the first and second images respectively assigned to the same heart phase is undertaken by a pixel-by-pixel subtraction of the first from the second images, wherein under some circumstances other linear combinations of the intensity values can also be computed, through which a series of phase-selective subtraction images is able to be created. Preferably the pixel intensities in the areas through which no blood is flowing are at least partly highlighted in the subtraction images, so that the blood vessels and the perfused tissue, or especially the contrast which is generated by the contrast medium, will be especially highlighted. Preferably the phase-selective subtraction images are free from movement artifacts and other destructive backgrounds. In other words only the areas of the blood vessels and of the tissue are represented in the subtraction images which are of interest for the subsequent examinations.

Preferably the phase-selective subtraction images are able to be used to create temporally and spatially resolved perfusion data of the heart. This typically enables time-density and/or time-intensity curves to be represented for individual pixels. Perfusion data such as blood volume, blood flow or mean transit time can be obtained from these curves for example and represented as a parameter map of the heart. As an alternative the second images can also be recorded in a state in which the contrast medium has already been distributed evenly in the vessels or the tissue, so that the second image is acquired in the “steady-state”. Spatially-resolved perfusion data can then also be obtained from the subtraction images, especially after a movement compensation, by registration of the series of subtraction images with one another.

Preferably the parameter map, which is also preferably able to be represented in gray values or also in color tones, is able to be represented on its own, also preferably an overlay of data obtained with other modalities is available, such as 3D x-ray data, 3D CT data or also magnetic resonance tomography data. Preferably the data of the other modalities is also able to be created in advance and is then registered with the recorded images.

A mask series also preferably comprises a complete heart cycle and a filling series at least two, preferably more than five, heart cycles. Preferably the mask series comprises at least one heart cycle such that each phase of the heart is recorded on the images. With the filling series it should be ensured that as many heart cycles are recorded as are needed for the contrast medium to have flowed into all vessels and tissue areas which are to be evaluated and to have flowed out again after the first pass, i.e. preferably both in the coronary arteries and also in the perfused heart tissue.

Also the number of first and second images recorded per second preferably correlates with the heart frequency determined by the assignment method. In other words the image frequency is thus able to be adapted to the heart frequency. In this way each phase and each point in time of the heart is able to be visually recorded. Preferably, with the heart rate of about 120 beats per minute with a given image frequency of approximately 60 images per second and an injection of a contrast medium of approximately 5 seconds, a sufficiently accurate temporal sampling is produced. As an alternative the method can also operate with a constant image frequency determined in advance or able to be set as variable.

Preferably the heart frequency is influenced by a frequency stimulation such that an even heart frequency is achieved. Preferably a heart phase control (cardiac pacing) is able to be applied, the heart frequency of which lies slightly above the body's own heart frequency, preferably at somewhat over 90 beats per minute. Preferably an upper limit of 120 beats per minute will not be exceeded. Cardiac pacing must generally occur by means of electrodes introduced into the heart, this is however possible since the contrast medium is also administered through intra-arterial catheters, so that in these cases an intervention in the patient is undertaken in any event. For patients with heart pacemakers said device takes over the cardiac pacing. Preferably the heart phase control in such cases is able to be applied both for the mask series and also for the filling series. The background to this procedure is basically that the heart cycle is able to be reproduced by said procedure, wherein the subtraction of the second images from the filling series from the first images from the mask series is facilitated. If necessary even the EKG can be omitted in this case, or only the starting point of each heart cycle must be determined using this procedure since it can be assumed that the further movement sequence in each cycle matches relatively precisely, that i.e. the heart, at a specific point in time of e.g. 30 ms after the QRS complex, has reached the same movement state in each case. The result is very high-quality phase-selective subtraction images.

Especially preferably the second state of the blood is initiated temporally by the assignment method. In other words the state in which the blood has the contrast medium added to it is thus able to be controlled temporally via the assignment method. In concrete terms the injection of the contrast medium bolus could be triggered at a specific point in time after the QRS complex in the EKG. Preferably the injection protocol is also adapted to the heart frequency measured by the assignment method or defined by the frequency stimulation, so that at least one complete heart cycle and thus the contrasting of the vessels and of the tissue by the contrast medium is covered. It is further preferred to define the beginning of the filling series with a specific delay after the injection, wherein the length of this delay can likewise depend on the measured heart frequency.

The blood vessels and/or the tissue are also preferably segmented in the second images and/or the phase-selective subtraction images and especially the center lines of the blood vessels are determined from the segmented areas. These are preferably represented parametrically or as a function, e.g. as a spline function. Known segmentation methods can preferably be employed in such cases, which are used for exact representation of the blood vessels and/or of the tissue. It is also preferred that the center lines are able to be used to align a patient ideally underneath the apparatus for the imaging method, for example a C-arm angiograph. Furthermore they can be used to optimize the trajectory of dynamic coronary scans, which observe the vessel from different angles, such as the COROSCAN procedures of the Artis Zee system for example.

Preferably a non-rigid registration of the second images or of the phase-selected subtraction images of a filling series between one another is carried out to create a series of movement-compensated, phase-selective subtraction images. This is advantageous since then, during the extraction of the time-density or time-intensity curves, the values which have been recorded at the same position in the tissue or in the vessel contribute to the curve in each case. Preferably during the registration two images following on from one another in time are registered to one another during the registration, i.e. one of the images is offset so that the corresponding areas again lie above one another in each case. Non-rigid registration methods are generally known.

In accordance with one embodiment the registration is undertaken on the basis of the center lines, i.e. the center lines are merely laid above one another in each case or their shift from image to image is computed. The tissue lying between them is defined elastically with specific characteristics and follows the center lines accordingly. As an alternative, specific regions of interest along the coronary arteries can also be used for this purpose, which can be readily detected on all images.

It is common to all registration methods that the heart movement pattern in the form of a movement vector field or in the form of transformation parameters will be obtained by the method. Such a movement vector field can preferably be computed from the images of a single heart cycle and then be used for the other heart cycles. The registration also allows time-density or time-intensity curves to be determined from the second images or the subtraction images for each pixel or for each position on the moving heart. From these curves in their turn perfusion parameters can be determined, which do not have any movement artifacts. In addition the movement vector fields or transformation parameters can be used also to dynamically represent the spatially-resolved perfusion data of the heart, i.e. dynamic flow representation. Advantageously they will however be used to compensate for the movement in the heart, in order for example to be able to represent the inflow of the contrast medium or corresponding perfusion parameters as a dynamic image or a parameter map, but without heart movement.

In accordance with a further embodiment the center lines or corresponding pixels of the phase-selected images are assigned via a movement vector field previously known as a model. The model maps a typical movement of the heart and thus also a specific area of interest, on which the examination is based. Preferably this known movement is present in the form of movement vectors, which are able to be assigned to the different pixels. It is thus possible to assign the images recorded at different points in time and the pixels contained therein to one another in that, on the basis of the movement vector of any given pixel, it can be calculated where it has to be located at a later point in time. The model can if necessary be adapted to the actual heart, i.e. by calculation of correlation factors between consecutive images and iterative corrections to the movement vector field.

In accordance with an alternative method of registration movement vectors for each pixel of the phase-selected images are determined by the pixel-by-pixel evaluation and approximation of time-intensity curves of the series of phase-selective subtraction images. This enables a model-based, non-rigid registration to be carried out. Preferably the time-intensity curves of each pixel of the phase-selective subtraction images are adapted to a Gamma variate function for this purpose. Through an iterative optimization process between this model (i.e. the Gamma variate function) and the actually-measured pixel intensities or densities (i.e. the dynamic perfusion curve) specific calculations (e.g. the Mean-Square-Difference) are carried out in each case in order to estimate the transformation parameters, i.e. the movement vector field of the heart. From the Gamma variate functions and the measured pixel intensities or their adaptation, a multi-dimensional optimization problem is produced which is preferably able to be solved with known algorithms. Movement vectors which are able to be used for the registration can be derived therefrom.

The registration is also preferably carried out for the center line and/or only for specific areas of the heart. Furthermore each area or each point of the heart is able to be used for the registration.

After use of the non-rigid registration preferably corresponding perfusion parameters are calculated from all second or subtraction images, and this is done without taking account of the heart phase, since the corresponding heart movement is compensated for by the movement vector field or the image transformation determined during the registration. The perfusion data is used for creating a perfusion parameter map.

Preferably the perfusion data is represented locally-resolved in a map of the heart. In this case the perfusion data can be represented phase-selectively and encoded as gray scales or colors in accordance with its value. In such cases a parameter map is preferably obtained, on which not only the coronary arteries are represented but also the profusion parameters of the perfused tissue. When the perfusion of the tissue is in focus, a longer injection duration can be selected.

Further, an apparatus is provided, which is a specially configured x-ray device. Preferably a magnetic resonance tomography device is also used.

An apparatus is specified for representing the blood vessels and/or the tissue in the heart of the human or animal body, comprising:
an imaging unit suitable for recording at least the series of temporally-resolved first and at least the series of temporary-resolved second images of the heart; an assignment unit which is suitable for assigning the first and second images to an associated heart phase in each case;
a calculation unit which is suitable for computing the first and second images such that a series of phase-selective subtraction images is able to be created;
wherein the computing unit is suitable, by computing the phase-selective subtraction images and/or the second images, taking into account the heart movement, for creating temporally and/or spatially-resolved perfusion data of the heart.

Especially preferably a C-arm angiograph is used, wherein the method may also able to be carried out with any other digital angiographic x-ray device.

An EKG unit is also preferably used as the assignment unit. The computing unit is preferably a correspondingly configured computer which is embodied so as to compute the first and second images, especially to subtract them from one another, in order to create phase-selective subtraction images.

The computing unit is also preferably configured so as to process the data of the assignment unit in order to assign the temporal information obtained therefrom to the image data. Expediently the computing unit is configured so as to enable it to create and to visualize the temporally and/or spatially-resolved perfusion data of the heart. Preferably a screen is able to be used for the visualization. Where the assignment unit does not have its own visualization unit the above-mentioned screen is preferably to be used for the visualization of the data of the assignment unit.

All variants described here are possible both in 2D and also in 3D.

Further advantages and features emerge from the subsequent description of preferred exemplary embodiments with reference to the enclosed figures, wherein individual features of individual embodiments can be combined into new embodiments.

### BRIEF DESCRIPTION OF THE DRAWINGS

FIG. 1 shows a schematic diagram of an apparatus in accordance with an embodiment,

FIG. 2 shows a schematic and stylized diagram of the creation of perfusion data of the heart and

FIG. 3 shows a flow diagram of a preferred method for the creation of perfusion data.

### DETAILED DESCRIPTION OF INVENTION

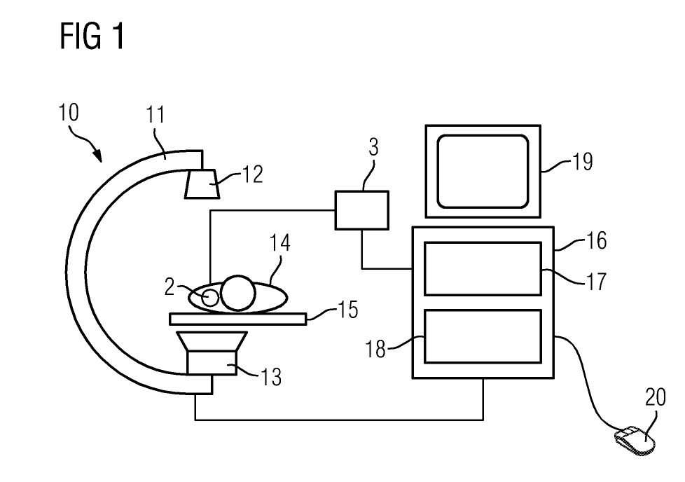

FIG. 1 shows a schematic diagram of a C-arm device which is able to be used for carrying out the described method and is configured for this purpose. The C-arm angiograph 10 has a C-arm 11, to the arms of which an x-ray tube 12 and a digital planar x-ray detector 13 are attached. The x-ray tube 12 can emit a cone of x-rays. The rays penetrate a body or a patient 14 lying on the patient support bed 15 and the non-absorbed radiation is detected by the planar detector 13 as a fluoroscopy or projection image 1, 1″. In the present case the rays especially penetrate the heart 2 shown in FIG. 1. The C-arm 11 is able to be rotated and moved with many degrees of freedom around the patient support bed 15, to enable images to be recorded from the largest possible number of projection angles. The images are transferred via a data line to the control and evaluation unit 16. This executes the computation steps of the method. For this purpose it generally has at least one storage device 17, for example a hard disk or optical disk, as well as a processing unit 18, for example a processor, especially a CPU and also random access memory (RAM), optionally an EPU and network connection. The control and evaluation unit 16 can involve a normal computer, especially a PC. This is preferably connected to a screen 19 which preferably displays the first images 1′ and/or the second images 1″ and/or the subtraction images 4 immediately after their creation, in order therewith to allow the doctor to control a diagnostic or surgical intervention. Furthermore an input device, such as a mouse 20 or a keyboard can be provided.

Furthermore the assignment unit 3, which is preferably embodied as an EKG, is used to carry out the method. Preferably the EKG contains units for acquisition of the EKG signal from the patient 14 and thus for representation of the heart cycle. Preferably the signal of the EKG is able to be forwarded to the processing unit 18 or to the control and evaluation unit 16, from where the EKG data is able to be computed and is if necessary visualized on the display 19.

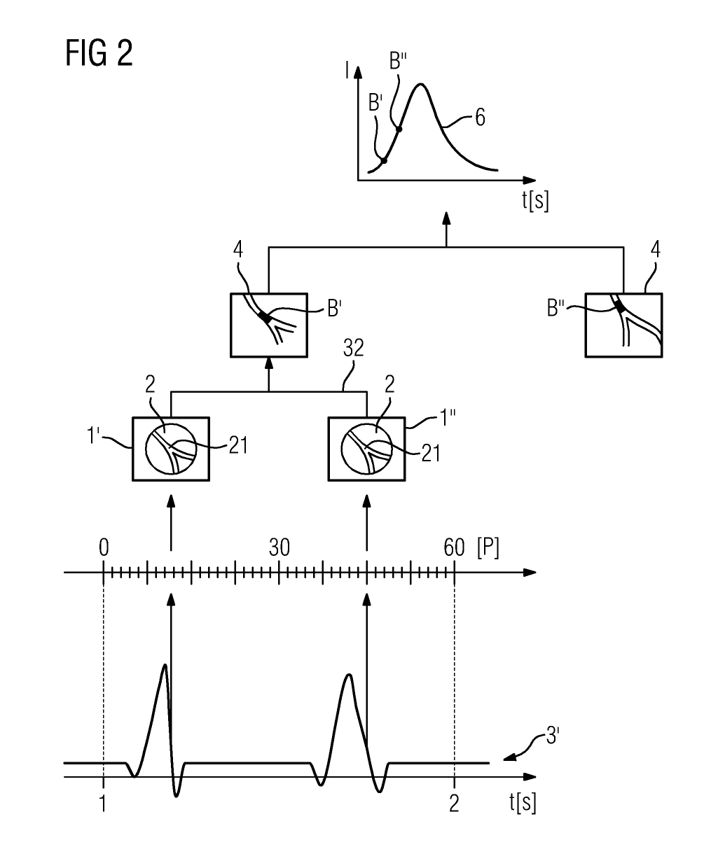

FIG. 2 visualizes for example the generation of perfusion data of the heart. The figure depicts an EKG signal plotted over time which is able to be determined by the assignment unit 3. A period of for example one second is shown. During this second 60 images (P) are recorded by the x-ray device. For example the images (P) of the first heart cycle are first images 1′, i.e. images of a mask series without contrast medium. The images of the second cycle shown in FIG. 2 are second images 1″ of a filling series with contrast medium.

The assignment unit 3 allows the first images 1′ and the second images 1″ to be assigned to the respective heart phase. In FIG. 2 this is depicted by the representation of the heart 2. Via a subtraction of the second images 1″ and the first images 1′ the blood vessels 21 are able to be explicitly represented in the movement-compensated, phase-selective subtraction image 4 for example. A pixel B′ is sketched out in FIG. 2. At a given time this pixel has a given density or intensity. At another/later point in time the same pixel will be designated as B″ and has a different intensity or density. Via a time-density function or the fitting of a Gamma variate function to the time-density function, the movement vectors of the different pixels can be computed from this.

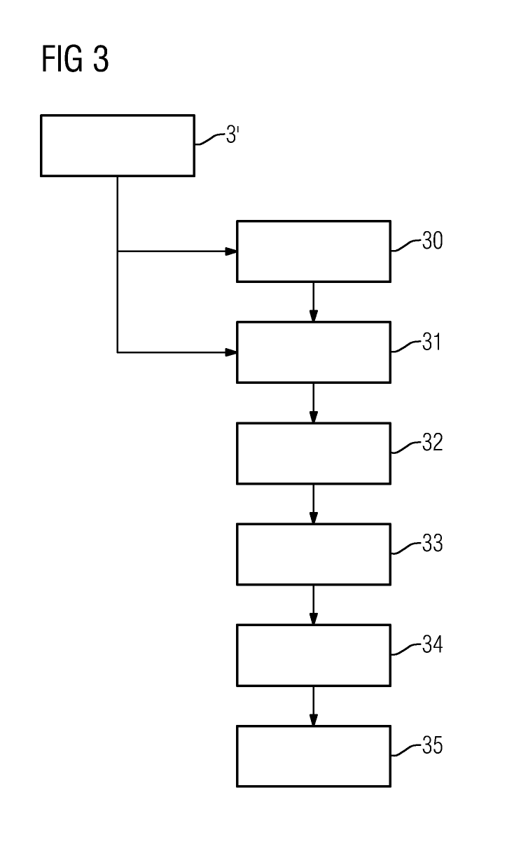

FIG. 3 shows a preferred practical flowchart for a preferred embodiment of the method. Initially a series of first images 1′ are acquired (30). Subsequently a series of second images 1″ are acquired (31). Both in the creation of the first images 1′ and also in the creation of the second images 1″ the EKG signal (3′) is recorded along with the image.

Subsequently the series of first 1′ and second images 1″ are computed (32) to create phase-selective subtraction images 4. This is followed by the segmentation (33) of the phase-selective subtraction images 4. This can be followed by the registration of the phase-selective subtraction images 4 to create (35) the movement-compensated temporally and spatially-resolved perfusion parameters.

While specific embodiments have been described in detail, those with ordinary skill in the art will appreciate that various modifications and alternative to those details could be developed in light of the overall teachings of the disclosure. For example, elements described in association with different embodiments may be combined. Accordingly, the particular arrangements disclosed are meant to be illustrative only and should not be construed as limiting the scope of the claims or disclosure, which are to be given the full breadth of the appended claims, and any and all equivalents thereof. It should be noted that the term “comprising” does not exclude other elements or steps and the use of articles “a” or “an” does not exclude a plurality.

1. A method for representing blood vessels and/or tissue of a heart of a human or animal body with an imaging method and an assignment method, which allows assignment of time-resolved images acquired with the imaging method to a heart phase, wherein blood in the blood vessels and/or tissue has two states, wherein the blood in the first state does not comprise a contrast medium, and wherein the blood in the second state comprises a contrast medium, the method comprising:
acquiring, by the imaging method, a mask series comprising temporally-resolved first images of the heart, wherein the blood is in the first state, and wherein the first images are assigned to a heart phase by the assignment method;
acquiring, by the imaging method, a filling series comprising temporally-resolved second images of the heart, wherein the blood is in the second state and wherein the second images are assigned to the heart phase by the assignment method;
computing the first and second temporally-resolved images assigned to the heart phase for creating phase-selective subtraction images;
computing the phase-selective subtraction images and/or the second images of at least one heart cycle, taking into account heart movement, for creating temporally and/or spatially-resolved perfusion data of the heart.

2. The method as claimed in claim 1, wherein the mask series covers at least one complete heart cycle and wherein the filling series covers at least two heart cycles.

3. The method as claimed in claim 1, wherein a number of first and second images recorded per second is correlated with a heart frequency determined by the assignment method.

4. The method as claimed in claim 3, wherein the heart frequency is influenced by a frequency stimulation such that an even heart frequency is provided.

5. The method as claimed in claim 1, wherein the second state of the blood is initiated temporally via the assignment method.

6. The method as claimed in claim 1, wherein the blood vessels and/or the tissue are segmented into the second images and/or the phase-selective subtraction images and wherein center lines of the blood vessels are determined based upon segmented areas.

7. The method as claimed in claim 6, wherein, in order to create movement-compensated, phase-selective subtraction images, a non-rigid registration of the second images or of the phase-selective subtraction images of a filling series is carried out.

8. The method as claimed in claim 7, wherein the registration is carried out on the basis of the center lines.

9. The method as claimed in claim 7, wherein corresponding pixels of the phase-selective subtraction images are assigned by a previously known movement vector.

10. The method as claimed in claim 7, wherein movement vectors for each pixel of the phase-selective subtraction images are determined through pixel-by-pixel evaluation and approximation of time density curves of the phase-selective subtraction images.

11. The method as claimed in claim 7, wherein the registration is carried out for the center lines and/or for specific areas of the heart.

12. The method as claimed in claim 1, wherein perfusion data is represented in a locally-resolved manner in a map of the heart.

13. An apparatus for representing blood vessels and/or tissue of a heart of a human or animal body, comprising:
an imaging device configured to image a series of time-resolved first images of the heart and a series of time-resolved second images of the heart;
an assignment unit configured to assign the first and second images to a heart phase;
a computing unit configured to compute the first and second images such that a series of phase-selective subtraction images is created;
wherein the computing unit is configured to created temporally and/or spatially-resolved perfusion data of the heat by computing the phase-selective subtraction images and/or the second images, taking into consideration the heart movement.
